(19) United States
(12) Patent Application Publication
(10) Pub. No.: US 2015/0065877 A1
(43) Pub. Date: Mar. 5, 2015

(54) METHOD AND SYSTEM FOR GENERATING A COMPOSITE ULTRASOUND IMAGE

(71) Applicant: General Electric Company, Schenectady, NY (US)

(72) Inventor: Fredrik Orderud, Oslo (NO)

(73) Assignee: General Electric Company, Schenectady, NY (US)

(21) Appl. No.: 14/015,355

(22) Filed: Aug. 30, 2013

(51) Int. Cl. A61B 8/08; A61B 8/06

(52) U.S. Cl. CPC A61B 8/5207; A61B 8/06
USPC 600/438

(57) ABSTRACT

A method and ultrasound imaging system includes acquiring first ultrasound data from a volume, acquiring second ultrasound data of a plane, the second ultrasound data including a different mode than the first ultrasound data. The method and system includes generating a composite image from both the first ultrasound data and the second ultrasound data, the composite image including a combination of a volume-rendering based on the first ultrasound data and a slice based on the second ultrasound data. The method and system includes displaying the composite image.

### FIELD OF THE INVENTION

This disclosure relates generally to a method and system for generating a composite image from different modes of ultrasound data.

### BACKGROUND OF THE INVENTION

It is possible to acquire many different modes of ultrasound data. Each mode of ultrasound data has its own unique set of strengths and weaknesses for a particular application. Two commonly used modes include B-mode and colorflow. B-mode, or brightness mode, assigns brightness values to pixels or voxels based on intensities of returning echoes. Colorflow, on the other hand, is a form of pulsed-wave Doppler where the strength of the returning echoes is displayed as an assigned color. Colorflow may be used to acquire velocity information on moving fluids, such as blood, or to acquire information on tissue movement. B-mode images are based on the acoustic reflectivity of the structures being imaged, while colorflow images indicate movement or velocity information. Both B-mode and colorflow images are very useful, but each mode conveys very different information.

B-mode images provide structural information regarding the anatomy being imaged. It is generally easy to identify specific structures and locations based on information contained in a B-mode image. Colorflow images, on the other hand, are used for assessing function within the body. A B-mode image does not convey the functional information contained in a colorflow image. A colorflow image, on the other hand, does not include as much information about structures and a patient's anatomy as a B-mode image. Using only a colorflow image, it may be difficult or impossible for a user to determine the exact anatomy corresponding to a particular portion of the colorflow image. Similar problems exist when viewing images generated based on other modes of ultrasound data as well.

For these and other reasons an improved method and ultrasound imaging system for generating and visualizing a composite image based on ultrasound data from two or more different ultrasound modes is desired.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes acquiring first ultrasound data from a volume and acquiring second ultrasound data of a plane. The second ultrasound data includes a different mode than the first ultrasound data. The method includes generating a composite image from both the first ultrasound data and the second ultrasound data. The composite image includes a combination of a volume-rendering based on the first ultrasound data and a slice based on the second ultrasound data. The method includes displaying the composite image.

In another embodiment, a method includes acquiring first ultrasound data of a volume and acquiring second ultrasound data from a plane intersecting the volume. The second ultrasound data includes a different mode than the first ultrasound data. The method includes generating a volume-rendering based on the first ultrasound data in a coordinate system. The method includes generating a slice based on the second ultrasound data in the coordinate system. The method includes merging the volume-rendering with the slice to generate a composite image and displaying the composite image.

In another embodiment, an ultrasound imaging system includes a probe, a transmitter coupled to the probe, a transmit beamformer coupled to the probe and the transmitter, a receive beamformer coupled to the probe, a display device, and a processor coupled to the probe, the transmitter, the transmit beamformer, the receive beamformer, and the display device. The processor is configured to control the transmitter, the transmit beamformer, the receive beamformer, and the probe to acquire first ultrasound data from a volume. The first ultrasound data includes a first mode. The processor is configured to control the transmitter, the transmit beamformer, the receive beamformer, and the probe to acquire second ultrasound data of a plane. The second ultrasound data includes a second mode. The processor is configured to generate a volume-rendering based on the first ultrasound data. The processor is configured to generate a slice based on the second ultrasound data. The processor is configured to generate a composite image including a combination of the volume-rendering and the slice. The processor is configured to display the composite image on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of geometry that may be used to generate a volume-rendering in accordance with an embodiment;

FIG. 3 is a flow chart illustrating a method in accordance with an embodiment;

FIG. 4 is a schematic representation of a volume and a slice from which ultrasound data may be acquired in accordance with an embodiment;

FIG. 5 is a schematic representation of a thick volume and a thin volume from which ultrasound data may be acquired in accordance with an embodiment;

FIG. 6 is a schematic representation of a composite image in accordance with an embodiment; and

FIG. 7 is a schematic representation of a composite image in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

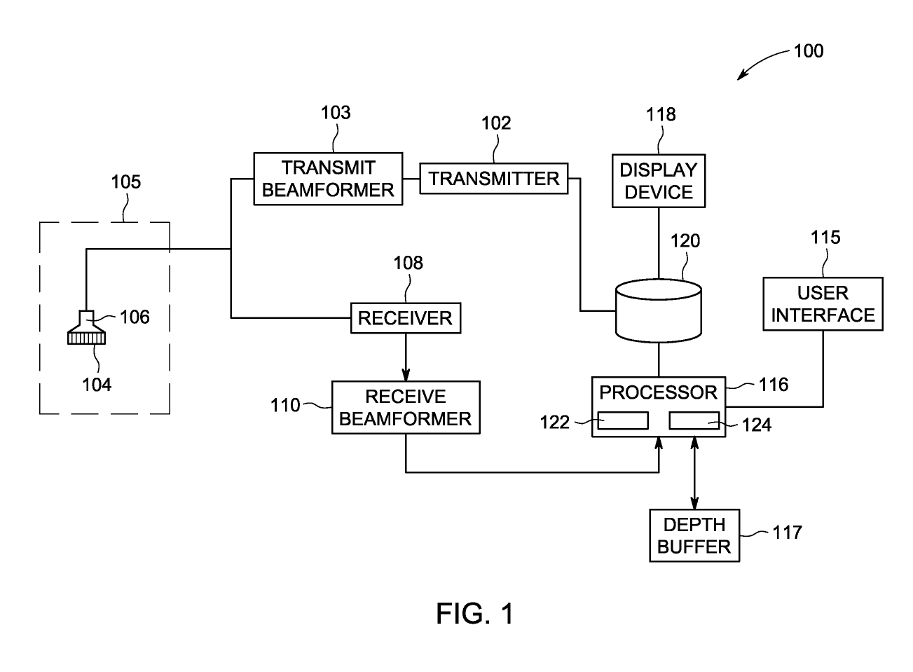

FIG. 1 is a schematic diagram of an ultrasound imaging system 100 in accordance with an embodiment. Couplings between various components of the ultrasound imaging system 100 are indicated on the schematic diagram by lines or arrows connecting the individual components. Each line or arrow may represent either a physical coupling, such as a wire or a fiber optic connection, or the lines may represent a wireless coupling between components. The lines or arrows represent the way the data or signals may travel through the various components of the ultrasound imaging system 100. The ultrasound imaging system 100 includes a transmitter 102 that transmits a signal to a transmit beamformer 103 which in turn drives transducer elements 104 within a transducer 106 to emit pulsed ultrasonic signals into a structure, such as a patient (not shown). A probe 105 includes the transducer 106 and the transducer elements 104. The probe 105 may be an electronically steerable 2D array according to an embodiment. According to other embodiments, the probe 105 may include a different configuration, including a mechanical 3D probe, or any other probe capable of acquiring volumetric data. The pulsed ultrasonic signals are back-scattered from structures in the body, like blood cells or muscular tissue, to produce echoes that return to the transducer elements 104. The echoes are converted into electrical signals by the transducer elements 104 and the electrical signals are received by a receiver 108. The electrical signals representing the received echoes are passed through a receive beamformer 110 that outputs ultrasound data. The ultrasound data may include 3D ultrasound data acquired from a volume, 2D ultrasound data acquired from a plane, or a plane reconstructed from a 3D ultrasound volume. A user interface 115 may be used to control operation of the ultrasound imaging system 100. For example, the user interface 115 may be used to control the input of patient data, to change a scanning or display parameter, to control the position of a 3D cursor, and the like.

The ultrasound imaging system 100 also includes a processor 116 to control the components of the ultrasound imaging system 100 and to process the ultrasound data for display on a display device 118. The processor 116 may include one or more separate processing components. For example, the processor 116 may include a graphics processing unit (GPU) according to an embodiment. Having a processor that includes a GPU may advantageous for computation-intensive operations, such as volume-rendering, which will be described in more detail hereinafter. The processor 116 may also include one or more modules, each configured to process received ultrasound data according to a specific mode. A first module 122 and a second module 124 are shown on FIG. 1 in accordance with an embodiment. Each module may include dedicated hardware components that are configured to process ultrasound data according to a particular mode. For example, the first module 122 may be a color-flow module configured to generate a color-flow image and the second module 124 may a B-mode module configured to generate a B-mode image. Other embodiments may not include separate modules within the processor 116 for processing different modes of ultrasound data. The processor 116 may be configured to implement instructions stored on a non-transitory computer-readable medium. The computer-readable medium may include any type of disk including floppy disks, optical disks, CD-ROMs, magneto-optical disks, ROMs, RAMs, EPROMs, EEPROMs, flash memory, magnetic or optical cards, or any other type of media suitable for storing electronic instructions.

The processor 116 is coupled to the transmitter 102, the transmit beamformer 103, the probe 105, the receiver 108, the receive beamformer 110, the user interface 115 and the display device 118. The processor 116 may be hard-wired to the aforementioned components or the processor 116 may be in electronic communication through other techniques including wireless communication. The display device 118 may include a screen, a monitor, a flat panel LED, a flat panel LCD, any other device configured to display a composite image as a plurality of pixels. The display device 118 may be configured to display images in stereo. For example, the display device 118 may be configured to display multiple images representing different perspectives at either the same time or rapidly in series in order to allow the user to view a stereoscopic image. The user may need to wear special glasses in order to ensure that each eye sees only one image at a time. The special glasses may include glasses where linear polarizing filters are set at different angles for each eye or rapidly-switching shuttered glasses which limit the image each eye views at a given time. In order to effectively generate a stereo image, the processor 116 may need to display the images on the display device 118 in such a way that the special glasses are able to effectively isolate the image viewed by the left eye from the image viewed by the right eye. The processor 116 may need to generate an image on the display device 118 including two overlapping images from different perspectives. For example, the first image from the first perspective may be polarized in a first direction so that it passes through only the lens covering the user's right eye and the second image from the second perspective may be polarized in a second direction so that it passes through only the lens covering the user's left eye.

The processor 116 may be adapted to perform one or more processing operations on the ultrasound data. Other embodiments may use multiple processors to perform various processing tasks. The processor 116 may also be adapted to control the acquisition of ultrasound data with the probe 105. The ultrasound data may be processed in real-time during a scanning session as the echo signals are received. For purposes of this disclosure, the term “real-time” is defined to include a process performed with no intentional lag or delay. The term “real-time” is further defined to include processes performed with less than 0.5 seconds of delay. An embodiment may update the displayed ultrasound image at a rate of more than 20 times per second. Ultrasound data may be acquired even as images are being generated based on previously acquired data and while a live or dynamic image is being displayed. Then, as additional ultrasound data is acquired, additional frames or images generated from more-recently acquired ultrasound data are sequentially displayed. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the ultrasound signal while a second processor may be used to further process the data prior to displaying an image. It should be appreciated that other embodiments may use a different arrangement of processors.

The processor 116 may be used to generate a volume-rendering from ultrasound data of a volume acquired by the probe 105. According to an embodiment, the ultrasound data may contain a value or intensity assigned to each of a plurality of voxels, or volume elements. In 3D ultrasound data, each of the voxels is assigned a value determined by the acoustic properties of the tissue or fluid corresponding to that particular voxel. The 3D ultrasound data may include B-mode data, color-flow data, strain mode data, tissue-velocity data, etc. according to various embodiments. The ultrasound imaging system 100 shown may be a console system, a cart-based system, or a portable system, such as a hand-held or laptop-style system according to various embodiments.

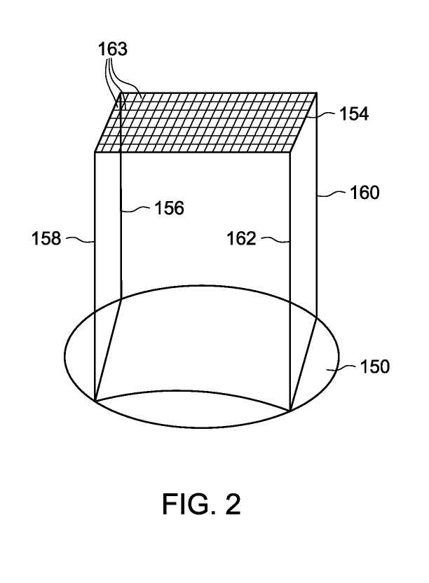

FIG. 2 is a schematic representation of geometry that may be used to generate a volume-rendering according to an embodiment. FIG. 2 includes 3D ultrasound data 150 and a view plane 154.

Referring to both FIGS. 1 and 2, the processor 116 may generate a volume-rendering according to a number of different techniques. According to an exemplary embodiment, the processor 116 may generate a volume-rendering through a ray-casting technique from the view plane 154. The processor 116 may cast a plurality of rays from the view plane 154 to the 3D ultrasound data 150. FIG. 2 shows ray 156, ray 158, ray 160, and ray 162 bounding the view plane 154. It should be appreciated that many more rays may be cast in order to assign values to all of the pixels 163 within the view plane 154. The 3D ultrasound data 150 comprises voxel data, where each voxel is assigned either an intensity and a depth value or an RGBA value and a depth value. According to an embodiment, the processor 116 may use a standard “front-to-back” technique for volume composition in order to assign a value to each pixel in the view plane 154 that is intersected by the ray. For example, starting at the front, that is the direction from which the image will be viewed, each voxel value along a ray is multiplied with its corresponding opacity value to form an opacity-weighted value. The opacity-weighted values are then accumulated in a front-to-back direction along each of the rays. This process is repeated for each of the pixels 163 in the view plane 154 in order to generate a volume-rendering. According to an embodiment, the pixel values from the view plane 154 may be displayed as the volume-rendering. The volume-rendering algorithm may be configured to use an opacity function providing a gradual transition from opacities of zero (completely transparent) to opacities of 1.0 (completely opaque). The volume-rendering algorithm may weigh the opacities of the voxels along each of the rays when assigning a value to each of the pixels 163 in the view plane 154. For example, voxels with opacities close to 1.0 will block most of the contributions from voxels further along the ray, while voxels with opacities closer to zero will allow most of the contributions from voxels further along the ray. Additionally, when visualizing a surface, a thresholding operation may be performed where the opacities of voxels are reassigned based on one or more threshold values. According to an exemplary thresholding operation, the opacities of voxels with values above a threshold may be set to 1.0 while voxels with opacities below the threshold may be set to zero. This type of thresholding eliminates the contributions of any voxels other than the first voxel above the threshold along the ray. Other types of thresholding schemes may also be used. For example, an opacity function may be used where voxels that are clearly above the threshold are set to 1.0 (which is opaque) and voxels that are clearly below the threshold are set to zero (translucent). However, an opacity function may be used to assign opacities other than zero and 1.0 to the voxels with values that are close to the threshold. This “transition zone” is used to reduce artifacts that may occur when using a simple binary thresholding algorithm. For example, a linear function mapping opacities to values may be used to assign opacities to voxels with values in the “transition zone.” Other types of functions that progress from zero to 1.0 may be used in accordance with other embodiments.

In an exemplary embodiment, gradient shading may be used to generate a volume-rendering in order to provide the user with a better perception of depth. For example, surfaces within the 3D ultrasound data 150 may be defined partly through the use of a threshold that removes data below or above a threshold value. Next, gradients may be defined at the intersection of each ray and the surface. As described previously, a ray is traced from each of the pixels 163 in the view plane 154 to the surface defined in the 3D ultrasound data 150. Once a gradient is calculated at each of the rays, a processor 116 (shown in FIG. 1) may compute light reflection at positions on the surface corresponding to each of the pixels and apply standard shading methods based on the gradients. According to another embodiment, the processor 116 identifies groups of connected voxels of similar intensities in order to define one or more surfaces from the 3D data. According to other embodiments, the rays may be cast from a single view point.

According to all of the non-limiting examples of generating a volume-rendering listed hereinabove, the processor 116 may use color in order to convey depth information to the user. Still referring to FIG. 1, as part of the volume-rendering process, a depth buffer 117 may be populated by the processor 116. The depth buffer 117 contains a depth value assigned to each pixel in the volume-rendering. The depth value represents the distance from the view plane 154 (shown in FIG. 2) to a surface within the volume represented in that particular pixel. A depth value may also be defined to include the distance to the first voxel with a value above that of a threshold defining a surface. Each depth value is associated with a color value according to a depth-dependent scheme. This way, the processor 116 may generate a color-coded volume-rendering, where each pixel in the volume-rendering is colorized according to its depth from the view plane 154. According to an exemplary colorization scheme, pixels representing surfaces at relatively shallow depths may be depicted in a first color, such as bronze, and pixels representing surfaces at deeper depths may be depicted in a second color, such as blue. The color used for the pixel may smoothly progress from bronze to blue with increasing depth according to an embodiment. It should be appreciated by those skilled in the art, that many other colorization schemes may be used in accordance with other embodiments.

Still referring to FIG. 1, the ultrasound imaging system 100 may continuously acquire ultrasound data at a frame rate of, for example, 5 Hz to 50 Hz depending on the size and spatial resolution of the ultrasound data. However, other embodiments may acquire ultrasound data at different rates. A memory 120 is included for storing processed frames of acquired ultrasound data that are not scheduled to be displayed immediately. In an exemplary embodiment, the memory 120 is of sufficient capacity to store at least several seconds of ultrasound data. The frames of ultrasound data are stored in a manner to facilitate retrieval thereof according to the order or time of acquisition. As described hereinabove, the ultrasound data may be retrieved during the generation and display of a live or dynamic image. The memory 120 may include any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well known by those skilled in the art and will therefore not be described in further detail.

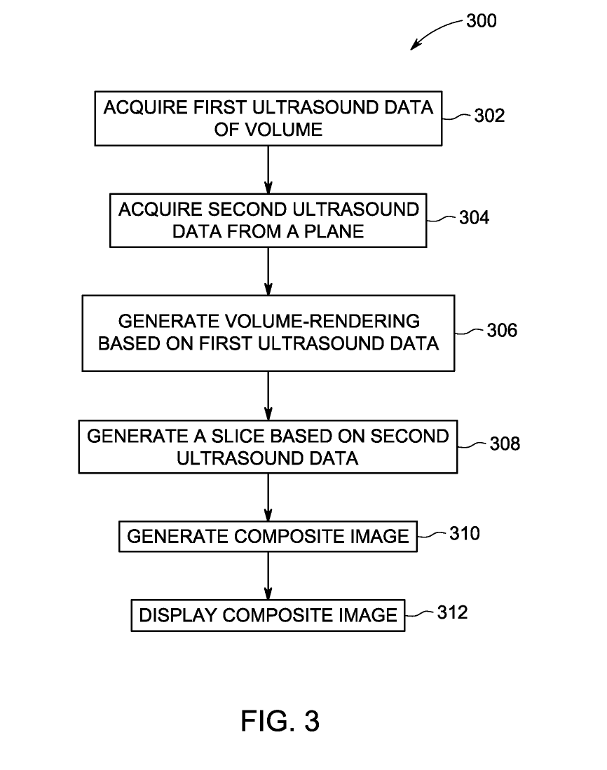

FIG. 3 is a flow chart illustrating a method 300 in accordance with an embodiment. The individual blocks represent steps that may be performed in accordance with the method 300. The technical effect of the method 300 is the display of a composite image including a combination of a volume-rendering and a slice, where the volume-rendering and the slice are generated from different modes of ultrasound data. The steps of the method 300 will be described according to an exemplary embodiment where the steps are performed with the ultrasound imaging system 100 (shown in FIG. 1).

Referring to both FIGS. 1 and 3, at step 302, the processor 116 controls the acquisition of first ultrasound data. The processor 116 controls the transmitter 102, the transmit beamformer 103, the probe 105, the receiver 108, and the receive beamformer 110 to acquire first ultrasound data in a first mode. According to an exemplary embodiment, the first mode may include a colorflow mode and the first ultrasound data may include colorflow ultrasound data acquired from a volume. It should be appreciated that the first ultrasound data may include ultrasound data of a different mode including B-mode data, tissue-velocity imaging data, strain data, as well as ultrasound data of any other mode.

At step 304, the processor 116 acquires second ultrasound data from a plane. According to an exemplary embodiment, the processor 116 controls the transmitter 102, the transmit beamformer 103, the probe 105, the receiver 108, and the receive beamformer 110 to acquire second ultrasound data in a second mode. The second ultrasound data may include B-mode data according to an exemplary embodiment. However, according to other embodiments, the second ultrasound data may include any other mode of ultrasound data including B-mode data, tissue-velocity imaging data, strain data, as well as ultrasound data acquired in any other mode. According to an exemplary embodiment, the plane may intersect through the volume from which the first ultrasound data was acquired. According to other embodiments, the second ultrasound data may include data acquired from two or more discrete planes. The planes may either intersect one another or they may be parallel to each other. According to yet other embodiments, the second ultrasound data may include volume data.

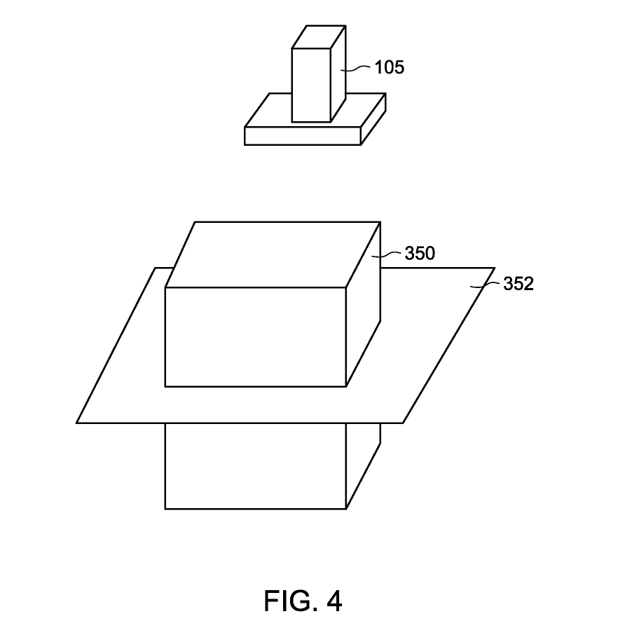

FIG. 4 is a schematic representation of a volume and a plane from which ultrasound data may be acquired according to an exemplary embodiment. The probe 105 from FIG. 1 is shown on FIG. 4 in accordance with exemplary acquisition geometry. Referring to the method 300 shown in FIG. 3, the first ultrasound data may be acquired from a volume 350. The volume 350 is a cuboid according to the embodiment shown in FIG. 3. However, it should be appreciated that the first ultrasound data may be acquired from volumes, or regions-of-interest, with different shapes according to other embodiments. As described with respect to step 302 of the method 300 (shown in FIG. 3), the processor 116 may control the ultrasound imaging system 100 to acquire ultrasound data of a first mode, such as color-flow data, from the volume 350.

FIG. 4 also includes a plane 352 intersecting the volume 350. During step 304 of the method 300 (shown in FIG. 3), the second ultrasound data may be acquired from one or more planes such as the plane 352. The second ultrasound data acquired from the plane 352 is of a different mode than the first ultrasound data acquired from the volume 350. For example, the second ultrasound data may be B-mode data. The plane 352 is shown as intersecting the volume 350 in FIG. 4. However, according to other embodiments, the second ultrasound data may be acquired from a plane that does not intersect the volume 350 from which the first ultrasound data was acquired. The second ultrasound data may include 2D ultrasound data of the plane 352, 2D ultrasound data of multiple planes, or the second ultrasound data may include 3D ultrasound data that includes the plane 352. One advantage of extracting image planes from 3D ultrasound data is that the plane 352 can be reconstructed in any direction, including directions oblique to the acquisition geometry.

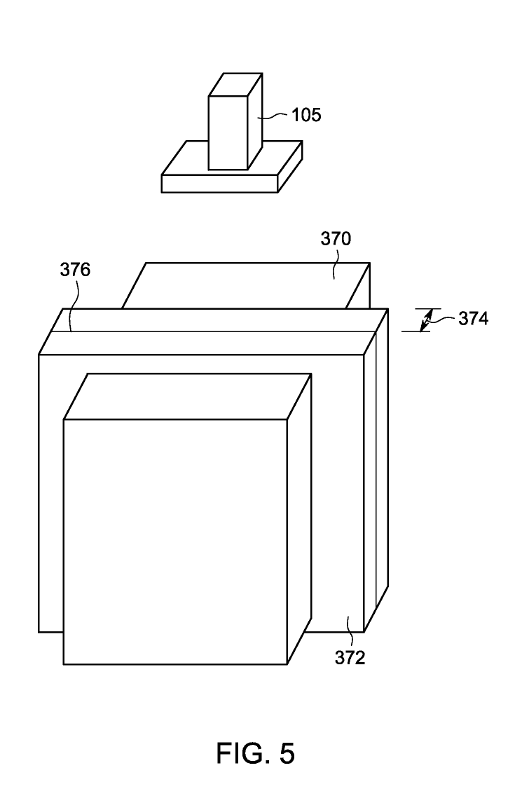

FIG. 5 is a schematic representation of a thick volume 370 and a relatively thin volume 372 from which ultrasound data may be acquired in accordance with an exemplary embodiment. The probe 105 from FIG. 1 is also shown. The thin volume 372 is positioned parallel with respect to the probe 105 for efficient acquisition. The thin volume 372 may be positioned in different orientations with respect to the probe 105 in other embodiments. The thin volume 372 has a thickness 374 and includes a plane 376 that is parallel to a side of the thin volume 372. According to an embodiment, the thin volume 372 may serve as a “thick plane.” That is, the data in the thin volume 372 may be collapsed in the direction of the thickness 374, so that the thin volume 372 becomes a plane. It should be appreciated that second ultrasound data may be acquired from planes other than those represented by the thin volume 372 (shown in FIG. 5).

Referring now to FIGS. 1, 3, and 4, at step 306 the processor 116 generates a volume-rendering based on the first ultrasound data acquired from the volume 350. An exemplary process of generating a volume-rendering was described hereinabove. The processor 116 may implement a similar process in order to generate the volume-rendering at step 306. The volume-rendering generated at step 306 will be the same mode as the mode of the first ultrasound data acquired at step 302. For example, if color-flow data was acquired at step 302, the volume-rendering generated from the first ultrasound data will be a color-flow volume-rendering. As part of generating the volume-rendering, the processor 116 may store a first plurality of depth-buffer values in a memory 120 or buffer. According to an embodiment, each pixel in the volume-rendering may be associated with a depth-buffer value representing the depth of the surface represented in that particular pixel of the volume-rendering.

Next, at step 308, the processor 116 generates a slice based on the second ultrasound data that was acquired at step 304. As previously described, the second ultrasound data may include either 2D data acquired from one or more planes, or the second ultrasound data may include data acquired from a volume. One or more slices may be reconstructed from the volume of data to represent various planes. The slice is the same mode as the second ultrasound data. According to an exemplary embodiment, the second ultrasound data may be B-mode ultrasound data and the slice would, therefore, be a B-mode representation of the plane 352. The slice may be either a 2D image or the representation of the slice may be a volume-rendering of the plane 352. As part of generating the slice, the processor 116 may store a second plurality of depth-buffer values in a memory or buffer. Each pixel in the slice may be associated with a depth buffer 117 value representing the depth of the portion of the slice represented by that particular pixel. If the second ultrasound data comprises 3D ultrasound data, then the second ultrasound data may already be in the same coordinate system as the volume-rendering. However, for other embodiments, it may be necessary for the processor 116 to convert the second ultrasound data into the same coordinate system as the volume-rendering. For example, the processor 116 may need to assign a depth-buffer value to each pixel in the slice in order to convert the second ultrasound data to voxel data of the same coordinate system as the first ultrasound data.

Referring back to FIG. 3, at step 310, the processor 116 generates a composite image. The composite image is based on both the volume-rendering generated at step 306 and the slice generated at step 308. As long as both the volume-rendering and the slice share a common coordinate system, it is possible for the processor 116 to merge the volume-rendering and the slice to form a composite image. The slice and the volume-rendering are represented in geometrically correct positions in the composite image. In other words, the position of the slice with respect to the volume-rendering in the composite image is the same as the position of the plane with respect to the volume from which the 3D ultrasound data was acquired. If the slice intersects the volume-rendering in the composite image, common anatomy will be represented in both the volume-rendering and the slice. The processor 116 may merge the volume-rendering with the slice according using several different techniques to manage regions where the slice and the volume-rendering overlap. However, it should be appreciated that the volume-rendering and the slice may not overlap in particular views of a composite image or in other embodiments.

According to a first embodiment, the processor 116 may combine the volume-rendering and the slice using a depth-buffer merge without alpha-blending. For example, the processor 116 may access the depth buffer 117 including the first depth-buffer values for the volume-rendering and the second depth buffer values for the slice and determine the proper spatial relationship between the slice and the volume-rendering based on the values in the depth buffer 117. Using a depth buffer 117 merge without alpha-blending may involve rendering surfaces with different depths so that the surface closest to the view plane 154 (shown in FIG. 2) is visible. According to an exemplary depth-buffer merge, the processor 116 may use the pixel value for whichever pixel is closer to the view plane 154 in order to generate the composite image. The processor 116 may implement an algorithm to determine whether or not to show the pixel value from the volume-rendering or the slice for each pixel location in the composite image.

According to another embodiment, the processor 116 may implement an alpha-blended merge in order to combine the volume-rendering with the slice. Each pixel in the volume-rendering and the slice may have an associated color and opacity. The processor 116 may implement an alpha-blended merge in order to combine pixel values from the volume-rendering and the slice in areas where the volume-rendering and the slice overlap. The processor 116 may combine pixels from the slice and the volume-rendering to generate new pixel values for the area of overlap including a blended color based on the volume-rendered pixel color and the slice pixel color. Additionally, the processor 116 may generate a summed opacity based on the opacity of the volume-rendered pixel and the opacity of the slice pixel. According to other embodiments, the composite image may be weighted to emphasize either the volume-rendering or the slice in either one or both of color and opacity. For example, the processor 116 may give more emphasis to either the value of the volume-rendered pixel or the slice pixel when generating the composite image.

According to another embodiment, both the first ultrasound data and the second ultrasound data may be voxel data in a common coordinate system. The processor 116 may combine the first ultrasound data with the second ultrasound data by combining voxel values in voxel space instead of first generating a volume-rendering based on the first ultrasound data and a slice based on the second ultrasound data. The first ultrasound data may be represented by a first set of voxel values and the second ultrasound data may be represented by a second set of voxel values. One or more values may be associated with each voxel such as color, opacity, and intensity. In B-mode ultrasound data, for example, an intensity representing the strength of the received echo signal is typically associated with each voxel, while in color-flow ultrasound data, a color representing the strength and direction of flow is typically associated with each voxel. Different values representing additional parameters may be associated with each voxel for additional types of ultrasound data. In order to combine the first ultrasound data and the second ultrasound data, the processor 116 may combine individual voxel values. The processor 116 may, for instance, combine or blend colors, opacities, or grey-scale values from the first set of voxel values with the second set of voxel values to generate a combined set of voxel values, or composite voxel data. Then, the processor 116 may generate a composite image by volume-rendering the composite voxel data. As with the previously described embodiment, the first ultrasound data may be weighted differently than the second ultrasound data when generating the composite image. According to another embodiment, the user may adjust the relative contribution of the first and second ultrasound data to the composite image in real-time based on commands entered through the user interface 115 (shown in FIG. 1).

Referring back to FIG. 3, at step 312, the processor 116 displays the composite image generated at step 310 on the display device 118.

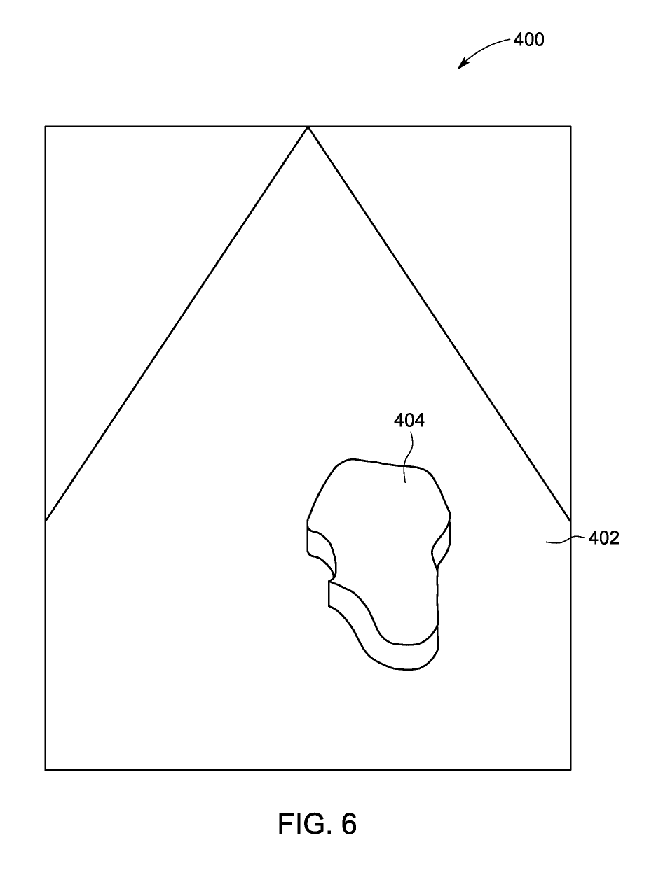

FIG. 6 is a schematic representation of a composite image 400 in accordance with an embodiment. The composite image 400 includes a slice 402 and a volume-rendering 404. According to an embodiment, the slice 402 may represent an image based on 2D ultrasound data from a plane. The volume-rendering 404 is superimposed over the slice 402. The volume-rendering 404 is based on 3D ultrasound data 150 and represents ultrasound data of a different mode than the slice 402. According to an embodiment, volume-rendering 404 may intersect the slice 402. For example, the slice 402 may intersect with the volume-rendering 404 along a plane. A region of the composite image 400 representing the intersection of the slice 402 and the volume-rendering 404 may be represented by pixels with blended intensities or colors (not shown in FIG. 6). The blended intensities or colors may be used to illustrate information from both the first ultrasound data and the second ultrasound data at the region of intersection. For example, according to an embodiment the region of intersection may include a color based on the first ultrasound data combined with a greyscale value based on the second ultrasound data, or a combination of colors and intensities from the first and second ultrasound data.

The user interface 115 (shown in FIG. 1) may be used to adjust the position of the slice 402 with respect to the volume-rendering 404. The slice 402, may, for example, be similar to a conventional 2D B-mode image. According to an embodiment, the composite image 400 represents ultrasound data acquired in real-time. The user may use the user interface 115 to adjust the position of the slice 402. For example, the user may adjust the angle of the slice 402 with respect to the volume-rendering 404, or the user may adjust the position of the slice 402 in any other direction, including a direction perpendicular to the slice 402. The position of the slice 402, and therefore the position of the plane from which ultrasound data is acquired to generate the slice 402, may be adjusted in real-time. The processor 116 may be configured to allow the user to position the slice 402 in any position with respect to the volume-rendering 404. The user may, for instance, position the slice 402 so that desired anatomy is visible and use the information in the slice 402 to better understand the data represented by the volume-rendering 404.

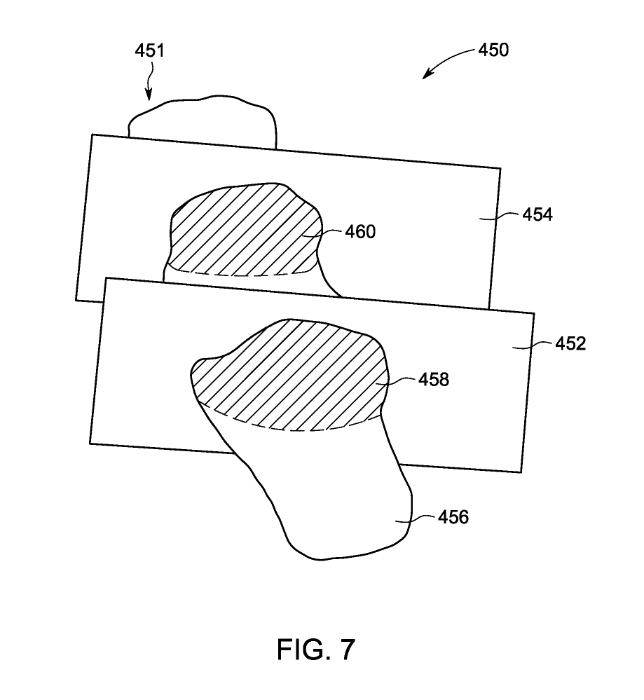

FIG. 7 is a schematic representation of a composite image 450 in accordance with an embodiment. The composite image 450 is a composite volume-rendering 451 including a first slice 452, a second slice 454, and a volume-rendering 456. For purposes of this disclosure, the term “composite volume-rendering” is defined to include a volume-rendering generated from at least two different modes of ultrasound data. The first slice 452 represents ultrasound data acquired from a first plane and the second slice 454 represents ultrasound data acquired from a second plane. The first slice 452 and the second slice 454 may both represent the same mode of ultrasound data, or the first slice 452 may be based on ultrasound data of a different mode than the second slice 454. Both the first slice 452 and the second slice 454 are shown intersecting the volume-rendering 456. The first slice 452 represents ultrasound data acquired from a first plane and the second slice 454 represents ultrasound data acquired from a second plane. The processor 116 (shown in FIG. 1) is adapted to adjust a view angle of the composite volume-rendering 451. For example, the composite volume-rendering 451 may be rotated and viewed from any direction. In addition, the position of one or both of the first slice 452 and the second slice 454 may be adjusted in real-time. The volume rendering 456 represents a first mode of ultrasound data, such as colorflow, while the first slice 452 and the second slice 454 both represent a second mode of ultrasound data such as B-mode. By rotating the composite volume-rendering 451, adjusting a level of zoom, and adjusting the positions of the slices 452, 454, a user is able to view slices at any position with respect to the volume-rendering 456. It should be appreciated that a different number of slices may be represented in other embodiments. According to an embodiment, the volume-rendering 456 may be based on colorflow data while the slices 452, 454 may be based on B-mode data. Viewing a composite image such as the composite image 450 provides an easy and intuitive way for a user to comprehend data acquired in multiple modes. According to an embodiment, a portion of the composite image 450 representing a region of intersection of the slices 452, 454 and the volume-rendering 456 may be represented by blending colors and intensities of the slices 452, 454 with the volume-rendering. A first region of intersection 458 and a second region of intersection 460 are represented with the hatching in FIG. 7. By adjusting the position of the slices 452, 454 with respect to the volume-rendering, the composite image 450 allows the user to easily understand the anatomy represented by a particular portion of the volume-rendering. Or, according to embodiments where the volume-rendering 456 represents anatomical data, such as B-mode data, the volume-rendering may be used to better understand the location of the data represented in the first slice 452 and the second slice 454.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for ultrasound imaging, the method comprising:
acquiring first ultrasound data from a volume;
acquiring second ultrasound data of a plane, the second ultrasound data comprising a different mode than the first ultrasound data;
generating a composite image from both the first ultrasound data and the second ultrasound data, the composite image comprising a combination of a volume-rendering based on the first ultrasound data and a slice based on the second ultrasound data; and
displaying the composite image.

2. The method of claim 1, wherein the first ultrasound data comprises color-flow data, strain data, or tissue-velocity imaging data; and the second ultrasound data comprises B-mode data.

3. The method of claim 1, wherein the composite image comprises a volume-rendering superimposed over at least a portion of the slice.

4. The method of claim 1, wherein the composite image comprises a composite volume-rendering of both the volume-rendering and the slice.

5. The method of claim 1, wherein the second ultrasound data comprises 2D ultrasound data of the plane.

6. The method of claim 1, wherein the second ultrasound data comprises data of a volume including the plane.

7. The method of claim 1, wherein the second ultrasound data compromises a first plane and a second plane that is distinct from the first plane, and wherein the composite image further comprises a second slice representing the second plane.

8. A method for ultrasound imaging, the method comprising:
acquiring first ultrasound data of a volume;
acquiring second ultrasound data from a plane intersecting the volume, the second ultrasound data comprising a different mode than the first ultrasound data;
generating a volume-rendering based on the first ultrasound data in a coordinate system;
generating a slice based on the second ultrasound data in the coordinate system;
merging the volume-rendering with the slice to generate a composite image; and
displaying the composite image.

9. The method of claim 8, wherein the volume-rendering includes first depth buffer values and the slice includes second depth-buffer values, and wherein said merging comprises merging the volume-rendering with the slice based on the first depth buffer values and the second depth buffer values.

10. The method of claim 8, wherein the first ultrasound data comprises color-flow data and the second ultrasound data comprises B-mode data.

11. The method of claim 8, wherein said generating the composite image comprises generating the composite image for display in stereo and said displaying the composite image comprises displaying the composite image in stereo.

12. The method of claim 8, wherein said generating the composite image comprises applying alpha-blending to a region of intersection representing overlap between the volume-rendering and the slice.

13. The method of claim 8, wherein said generating the composite image comprises applying a z-buffer merge to a region of intersection representing the intersection of the slice and the volume-rendering.

14. The method of claim 8, further comprising automatically updating the composite image in response to adjusting a position of the plane.

15. The method of claim 8, further comprising independently adjusting an opacity of the slice or of the volume-rendering in the composite image.

16. An ultrasound imaging system, the system comprising:
a probe;
a transmitter coupled to the probe;
a transmit beamformer coupled to the probe and the transmitter;
a receive beamformer coupled to the probe;
a display device; and
a processor coupled to the probe, the transmitter, the transmit beamformer, the receive beamformer, and the display device, wherein the processor is configured to:
control the transmitter, the transmit beamformer, the receive beamformer, and the probe to acquire first ultrasound data from a volume, the first ultrasound data comprising a first mode;
control the transmitter, the transmit beamformer, the receive beamformer, and the probe to acquire second ultrasound data of a plane, the second ultrasound data comprising a second mode;
generate a volume-rendering based on the first ultrasound data;
generate a slice based on the second ultrasound data;
generate a composite image comprising a combination of the volume-rendering and the slice; and
display the composite image on the display device.

17. The ultrasound imaging system of claim 16, wherein the processor comprises a first module configured to generate the volume-rendering and a second module configured to generate the slice.

18. The ultrasound imaging system of claim 17, wherein the first module comprises a color-flow module and the second module comprises a B-mode module.

19. The ultrasound imaging system of claim 16, further comprising a user interface, and wherein the processor is further configured to adjust a position of the plane in response to a command entered through the user interface.

20. The ultrasound imaging system of claim 19, wherein the processor is further configured to update the composite image and display the updated composite image in response to the command adjusting the position of the plane.

21. The ultrasound imaging system of claim 16, wherein the processor is configured to adjust the view angle and zoom of the composite image on the display device.

22. The ultrasound imaging system of claim 16, wherein the processor is configured to generate the composite image for display in stereo and the display device is adapted to display the composite image in stereo.
